(19) United States
(12) Patent Application Publication
(10) Pub. No.: US 2023/0273192 A1
(43) Pub. Date: Aug. 31, 2023

(54) FLUORESCENT COMPOUND FOR DETECTING BIOLOGICAL MATERIAL AND PREPARATION METHOD THEREOF

(71) Applicant: BIOACTS CORPORATION, Incheon (KR)

(72) Inventors: JIN WOO PARK, Incheon (KR); KIWON KIM, Incheon (KR); SU JUNG JANG, Incheon (KR); NURI CHOI, Incheon (KR); GYEONG RIM SHIN, Incheon (KR)

(21) Appl. No.: 18/073,631

(22) Filed: Dec. 2, 2022

(30) Foreign Application Priority Data
Feb. 27, 2022 (KR) 10-2022-0025611

(51) Int. Cl. G01N 33/533; C07D 405/14; C07D 405/12; C07D 491/153

(52) U.S. Cl. CPC G01N 33/533; C07D 405/12; C07D 405/14; C07D 491/153

(57) ABSTRACT

Provided is a fluorescent compound for detecting a biomaterial having the following Chemical Formula 1:

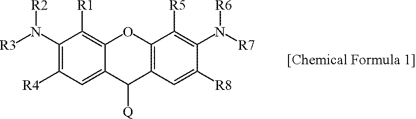

In Chemical Formula 1 above,
R₁ and R₅ are the same as or different from each other, and each independently selected from H, —SO₃⁻ and SO₃H,
R₂ and R₆ are the same as each other or each independently selected from hydrogen or methyl
R₄ and R₈ are the same as or different from each other, and each independently selected from H, —SO₃⁻ and (CH₂)SO₃H,
R₃ and R₄ can form alkylene chain with arbituary-substituted 3 carbons combined with the nitrogen atom attached with R₃,
R₇ and R₈ can form alkylene chain with arbituary-substituted 3 carbons combined with the nitrogen atom attached with R₇,
when R₈ and R₈ do not form alkylene chain, R₆, R₇ and R₈ are hydrogen at the same time and the nitrogen atom attached with R₆ and R₇ at the same time forms double bond with the carbon of mother molecule and has positive charge.

### CROSS REFERENCE TO RELATED APPLICATIONS

This application claims priority from and the benefit of Korean Patent Application No. 10-2022-0025611, filed on Feb. 27, 2022, which is hereby incorporated by reference for all purposes as if fully set forth herein.

### BACKGROUND

### Field

The present disclosure relates to a fluorescent compound, which is a compound that is useful for a fluorescent diagnosis composition capable of prediction, diagnosis, treatment and prognosis of diseases.

The fluorescent compound provided in exemplary embodiments of the present invention relates to a fluorescent compound which improves that the fluorescence efficiency of conventional rhodamine compounds is not high and comprises a triazine-substituted benzene linker to a rhodamine-based compounds.

The fluorescent compound provided in exemplary embodiments of the present invention has less noise and high fluorescent efficiency to improve the efficiency of a fluorescent signal when a desired biomaterial is detected using a fluorescence diagnostic composition of the present invention and thus can more accurately diagnose the biomaterial than the related art.

### Description of the Related Art

Since a biomaterial itself has weak fluorescence or no fluorescence in visible and near-infrared regions, in a bio field, in order to observe biological phenomena at cellular and subcellular levels in vivo or in vitro or to make images and obtain optical images of a diseased area by being projected into a living body, imaging data have been obtained through a variety of methods using a fluorescent dye or a specific biomaterial pre-labeled with the fluorescent dye in the biomaterial with optical equipment.

Various optical analysis devices used in the bio field select a fluorescent dye with an excitation wavelength and an emission wavelength suitable for observing fluorescence according to embedded light sources and filters as a basic material or reagent.

In general, most of fluorescent dyes used for labeling biomolecules such as proteins or peptides include structures, such as anthranilate, 1-alkylthic isoindoles, pyrrolinones, bimanes, benzoxazole, benzimidazole, benzofurazan, naphthalenes, coumarins, cyanine, stilbenes, carbazoles, phenanthridine, anthracenes, bodipy, fluoresceins, eosins, rhodamines, pyrenes, chrysenes and acridines.

When selecting a fluorescent dye structure usable in the bio field from a plurality of fluorescent chromophores illustrated above, generally, it is important to emit strong fluorescence when most of biomolecules exist in a medium, that is, an aqueous solution and an aqueous buffer, and to have excitation and fluorescence wavelengths suitable for fluorescence equipment.

Dyes that may be mainly applied in the bio field need to preferably have less photobleaching and quenching in aqueous or hydrophilic conditions, to have a large molecular extinction coefficient so as to absorb a large amount of light, to be in the visible or near-infrared region of 500 nm or more far from the fluorescence range of the biomolecule itself, and to be stable under various pH conditions. However, structures of dyes usable for labeling biomolecules capable of satisfying the limitations are limited.

Fluorescent chromogens that meet these requirements include cyanine, rhodamine, flocseine, bodipy, coumarin, acridine, and pyren derivatives, and introduce functional groups so as to bind to a dye alone or a specific substituent in a biomolecular structure. Among them, xanthane-based flocseine and rhodamine, and polymethine-based cyanine derivative dye compounds are mainly commercialized.

In particular, the dye compound having the cyanine chromophore has an advantage that it is easy to synthesize compounds various absorption/excitation wavelengths. In addition, generally, since the dye compound having the rhodamine chromophore is excellent in optical and pH stability, has narrow absorption and emission wavelength ranges, and has a fluorescent area of 400 to 600 nm, the dye compound is not overlapped with the self-fluorescent region of the biomolecule to be easily analyzed and has a slight difference according to a solvent and solubility characteristics, but has many advantages such as representing high molar adsorption coefficient, and thus is frequently used for biological applications.

In addition, the dye compound having the rhodamine chromophore may also be usefully used for optical filters for image display devices or resin compositions for laser fusion. The compound having a large intensity of absorption in specific light has been widely used as an optical element of an optical filter for an image display device such as a liquid crystal display device, a plasma display panel, an electroluminescence display, a cathode tube display panel, and a fluorescent display tube or an optical recording medium of DVD±R and the like. The optical filter has required a function of selectively absorbing light having unnecessary wavelengths, and simultaneously has required light absorption of wavelengths of 400 to 600 nm to prevent reflections or glare of external light such as fluorescent light, and has required a function of selectively absorbing wavelengths of infrared light in order to increase the image quality.

Therefore, in order to usefully apply the dyes industrially, it has been continuously required to develop novel dyes that have excellent optical and pH stability, have a narrow absorption/emission wavelength range in a specific wavelength range, and exhibit a high molar absorption coefficient.

### SUMMARY

An object of the present invention is to provide a fluorescent compound, a preparation method of the compound or a fluorescent diagnostic composition including the compound capable of being used as a contrast medium composition by improving further the fluorescent intensity in a florescent region of 400 to 600 nm while having excellent optical and pH stability and a narrow absorption/emission wavelength range, and particularly, improving the fluorescence by introducing a benzene linker having a triazine structure to a rhodamine-based fluorescent compound.

In order to solve the above-described problems, the inventors of the present application have developed a fluorescent compound represented by the following Chemical Formula 1.

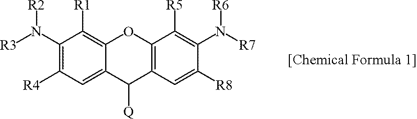

In Chemical Formula 1 above,
R₁ and R₅ are the same as or different from each other, and each independently selected from H, —SO₃⁻ and SO₃H,
R₂ and R₆ are the same as each other or each independently selected from hydrogen or methyl
R₄ and R₈ are the same as or different from each other, and each independently selected from H, —SO₃⁻ and (CH₂)SO₃H,
R₃ and R₄ can form alkylene chain with arbituary-substituted 3 carbons combined with the nitrogen atom attached with R₃,
R₇ and R₈ can form alkylene chain with arbituary-substituted 3 carbons combined with the nitrogen atom attached with R₇,
when R₈ and R₈ do not form alkylene chain, R₆, R₇ and R₈ are hydrogen at the same time and the nitrogen atom attached with R₆ and R₇ at the same time forms double bond with the carbon of mother molecule and has positive charge,
the said Q is a substituent having a chemical structure below

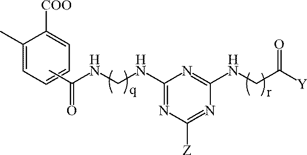

Z is OH or NH(CH₂)ₛSO₃H,
q is an integer of 0 to 10,
r is an integer of 1 to 10, and
s is an integer of 1 to 7,
Y is selected from H, an N-succinimidol group, a hydrazinyl group, an N-hydroxysuccinimidyl group, an N-hydroxysuccinimidyl oxy group, a sulfosuccinimidyl oxy, a 4-sulfo-2,3,4,5-tetrafluoro phenyl group, a maleicimide C₀-₁₀ alkylamyl group, a vinylsulfonyl group, a vinylsulfonyl C₀-₆ alkylaminyl group and an amino C₀-₆ alkyl.

According to exemplary embodiments of the present invention, the fluorescent compound has high stability under a water-soluble condition to be easily stored for a long time and improve pH stability, and particularly, can be more efficiently used for labeling and dyeing of a target material to improve the fluorescent intensity even at a low concentration as compared with the conventional structure. Further, the fluorescent compound is excellent in optical stability and exhibits stable fluorescence in long-term dyeing, and is excellent in fluorescence intensity while being not accumulated in the body, and thus, can be easily dyed and imaged in vivo even in the use of a small amount as compared with the conventional dyes to be economically used.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 shows the absorbance fluorescent spectrum of the compound 2 in accordance with embodiments of the present invention and a conventional dye.

FIG. 2 shows the optical characteristics of the compound 2 in accordance with embodiments of the present invention and a conventional dye.

FIG. 3 shows the labeling ratios of the compound 2 and a conventional dye by the amount of reacted-dye.

FIG. 4 shows the graph of the labeling ratios of the compound 2 and a conventional dye by the amount of reacted-dye.

FIG. 5 shows the fluorescent intensity of the compound 2 and a conventional dye by the ratio of reacted-dye.

FIG. 6 shows the fluorescent intensity of the compound 2 and a conventional dye by the labeling ratio.

FIG. 7 shows the absorption intensity in the same molar concentration of the compound 4 in accordance with embodiments of the present invention and a conventional dye.

FIG. 8 shows the fluorescent intensity in the same molar concentration of the compound 4 in accordance with embodiments of the present invention and a conventional dye.

FIG. 9 shows the fluorescent intensity in the same weight concentration of the compound 4 in accordance with embodiments of the present invention and a conventional dye.

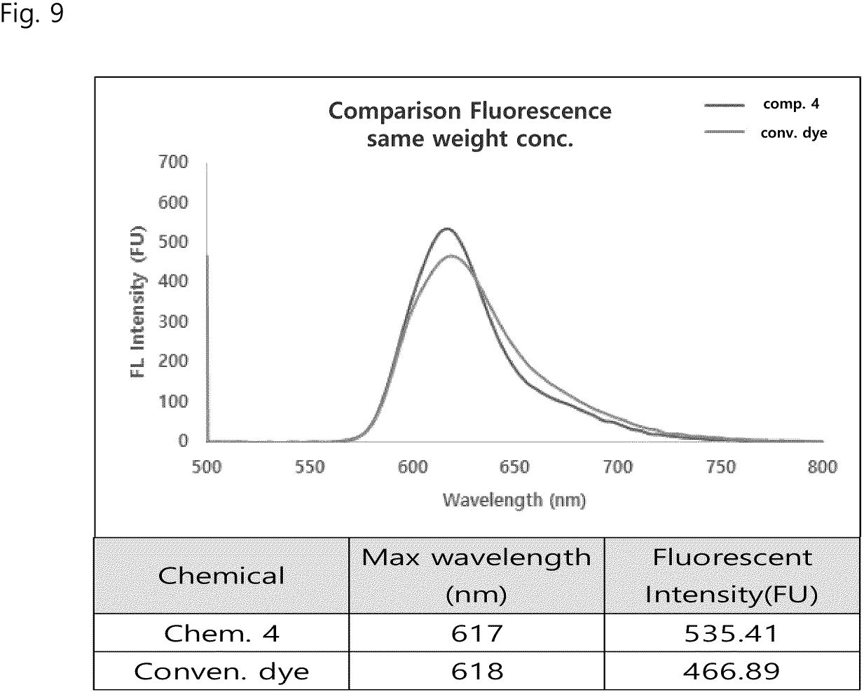

FIG. 10 shows the fluorescent intensity in the same absorption intensity of the compound 4 in accordance with embodiments of the present invention and a conventional dye.

FIG. 11 shows the labeling ratios of the compound 4 and a conventional dye by the amount of reacted-dye.

FIG. 12 shows the graph of the labeling ratios of the compound 4 and a conventional dye by the amount of reacted-dye.

FIG. 13 shows the fluorescent intensity of the compound 2 and a conventional dye by the ratio of reacted-dye.

FIG. 14 shows the fluorescent intensity of the compound 2 and a conventional dye by the labeling ratio.

FIG. 15 shows the ability of Immunofluorescence staining of the compounds in accordance with embodiments of the present invention and a conventional dye.

### DETAILED DESCRIPTION OF THE PREFERRED EMBODIMENT

A fluorescent compound in accordance with exemplary embodiments of the present invention is invented to improve the problem that the fluorescent dye having aminosulfonic trizine has a lot of noise and the fluorescent effectiveness is low of that kind of dye. The present invention is a rhodamine-based fluorescent dye in which the benzene linker substituted by triazine introduced as a linker to solve the problem.

Hereinafter, preparation methods of a fluorescent compound and a surfactant compound in accordance with exemplary embodiments of the present invention and the fluorescence efficiency of the composition in accordance with exemplary embodiments of the present invention will be described in detail by using embodiments of the present invention.

Hereinafter, the present invention will be described in more detail through Examples of the present invention. However, the following Examples are not to limit the scope of the present invention and will be described to help in the understanding of the present invention.

Exemplary embodiments of the present invention may use a fluorescent compound represented by the following Chemical Formula 1.

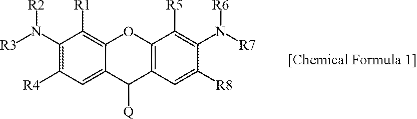

In Chemical Formula 1 above,
R₁ and R₅ are the same as or different from each other, and each independently selected from H, —SO₃⁻ and SO₃H,
R₂ and R₆ are the same as each other or each independently selected from hydrogen or methyl
R₄ and R₈ are the same as or different from each other, and each independently selected from H, —SO₃⁻ and (CH₂)SO₃H,
R₃ and R₄ can form alkylene chain with arbituary-substituted 3 carbons combined with the nitrogen atom attached with R₃,
R₇ and R₈ can form alkylene chain with arbituary-substituted 3 carbons combined with the nitrogen atom attached with R₇,
when R₈ and R₈ do not form alkylene chain, R₆, R₇ and R₈ are hydrogen at the same time and the nitrogen atom attached with R₆ and R₇ at the same time forms double bond with the carbon of mother molecule and has positive charge,
the said Q is a substituent having a chemical structure below

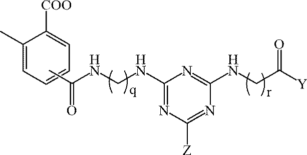

Z is OH or NH(CH₂)ₛSO₃H,
q is an integer of 0 to 10,
r is an integer of 1 to 10, and
s is an integer of 1 to 7,
Y is selected from H, an N-succinimidol group, a hydrazinyl group, an N-hydroxysuccinimidyl group, an N-hydroxysuccinimidyl oxy group, a sulfosuccinimidyl oxy, a 4-sulfo-2,3,4,5-tetrafluoro phenyl group, a maleicimide C₀-₁₀ alkylamyl group, a vinylsulfonyl group, a vinylsulfonyl C₀-₆ alkylaminyl group and an amino C₀-₆ alkyl.

The compound of Chemical Formula 1 provided in exemplary embodiments of the present invention may be useful to detect the biomaterial by labeling the biomaterial, and the biomaterial may be selected from the group consisting of proteins, peptides, carbohydrates, sugars, fats, antibodies, proteoglycan, glycoprotein, and siRNA.

Further, when labeling the biomaterial, the fluorescent compound provided in exemplary embodiments of the present invention binds to at least one functional group selected from an amine group, a hydroxyl group, and a thiol group in the biomaterial to label the biomaterial.

The method for labeling the fluorescent compound represented by Chemical Formula 1 is performed by using a buffer selected from the group consisting of a phosphate buffer, a carbonate buffer, and a tris buffer, an organic solvent selected from the group consisting of dimethyl sulfoxide, dimethylformamide, methanol, ethanol and acetonitrile, or water as a solvent and reacting the biomaterial, nanoparticles, or organic compounds with the compound of Chemical Formula 1 at pH 5 to 12. The reaction may be carried out for 30 minutes to 48 hours at a temperature of 20° C. to 80° C.

Most of biomaterials are dissolved in a predetermined buffer from a packaging unit, and in many cases, a separate buffer or pH is required to secure the stability of the biomaterials, and as a result, it is not easy to adjust the buffer or pH with a variable. The compound of Chemical Formula 1 according to exemplary embodiments of the present invention reacts reacting with proteins in various buffers, reaction temperatures, and pH conditions to express fluorescence and thus, is suitable to be used for labeling the biomaterials.

A preparation method of the compounds included in Chemical Formula 1 will be described.

### Example 1: Synthesis of Initial Compounds A-1 and A-4 for Preparing Compounds in Accordance With Exemplary Embodiments of Present Invention

### Synthesis of Compound A-1

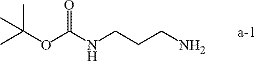

1,3-diaminopropane (20 g, 270 mmol, 7.96 eq) was dissolved in 70 ml of 1,4-dioxane. Di-tert-butyl dicarbonate (7.4 g, 33.9 mmol, 1 eq) was dissolved in 70 ml of 1,4-dioxane and then trickled in a 1,3-diaminopropane solution, and stirred at room temperature day and night and then dried under reduced pressure. The dried material was dissolved in distilled water and then filtered to extract the obtained filtrate with methylene chloride three times. An organic layer obtained after extraction was dried under reduced pressure to obtain a compound 3-1. (6 g, 91.5%)

R_{f} = 0.4 (Silicagel, methylene chloride:methanol = 8:1)

### Synthesis of Compound A-2

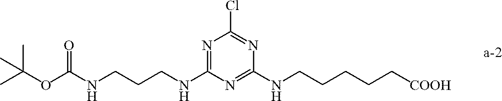 [SMILES: CC(C)(C)OC(=O)NCCCNc1nc(Cl)nc(NCCCCCC(=O)O)n1]

A compound a-1 (5.1 g, 29.27 mmol, 1 eq) was dissolved in a mixed solution of 150 ml of acetone and 50 ml of distilled water and then stored at 4° C. or less. Cyanuric chloride (CNC) (5.4 g, 29.27 mmol, 1 eq) was fully dissolved in 150 ml of acetone and then added with 50 g of ice and dispersed at 4° C. or less. The compound a-1 solution was trickled in a CNC solution, and then trickled in an aqueous solution of sodium hydrogencarbonate (fully dissolving 2.46 g carbonate in 50 ml of distilled water) and then, the reaction was performed at 4° C. or less for 2 hours. 6-aminohexanoic acid (1.42 g, 29.27 mmol, 1 eq) was dissolved in 50 ml of distilled water and then trickled in the reaction solution. The aqueous solution of sodium hydrogencarbonate was trickled and the reaction was performed at room temperature for 2 hours and then stirred at 40° C. day and night. The reaction solution was dried under reduced pressure and purified using silica gel chromatography to obtain a compound 3-2. (9 g, 73.8%)
R_{f} = 0.7 (Silicagel, methylene chloride:methanol = 8:1)
LC/MS, calculated value of C₁₇H₂₉ClN₆O₄ 416.91, measured value of 415.2

### Synthesis of Compound A-3

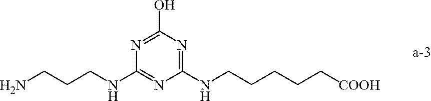 [SMILES: NCCCNc1nc(O)nc(NCCCCCC(=O)O)n1]

The compound a-2(3 g, 7.2 mmol, 1 eq) was fully dissolved in 40 ml of acetonitrile(ACN) and then added with 40 ml of distilled water. Thereafter 6N hydrochloric acid 20 ml was added and the reaction was performed at 60° C. for a day and night. The reaction solution was lyophilized and subjected to a reverse phase column to obtain a compound a-3. (1.5 g, 69.8 %)
R_{f} = 0.4 (Silicagel, methylene chloride:methanol = 8:1)
LC/MS, calculated value of C₁₂H₂₂N₆O₃ 298.35, measured value of 297.3

### Synthesis of Compound A-4

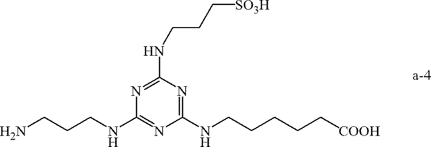 [SMILES: NCCCNc1nc(NCCCCCC(=O)O)nc(NCCCS(=O)(=O)O)n1]

Compound a-2 (4 g, 9.61 mmol, 1 eq) and 3-Amino-1-propanesulfonic acid (1.6 g, 11.53 mmol, 1.2 eq) were fully dissolved in 6.7 ml Dimethylformamide(DMF) and then added with 40 ml of distilled water.

Thereafter 2 ml of 30% sodium hydroxide was added and then stirred in 4 hours at 100° C. and the reaction-solution was lyophilized.

Thereafter, 40 ml of a 6 N aqueous hydrochloric acid solution was added, and then the solution was reacted for 2 hours at room temperature. The reaction solution was lyophilized and subjected to a reverse phase column to obtain a compound a-4. (1.6 g, 40 %)
R_{f} = 0.23 (Silicagel, methylene chloride:methanol = 8:1)
LC/MS, calculated value of C₁₅H₂₉N₇O₅S 298.35, measured value of 419.50

### Example 2: Synthesis of Compound 1 With Exemplary Embodiments of the Present Invention

### Synthesis of Compound B-1

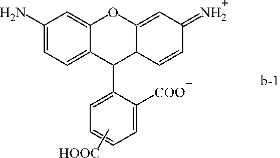

Trimellitic acid (13 g, 0.062 mol, 1 eq, TCI) and 3-Aminophenol (20 g, 0.186 mmol, 3 eq, TCI) were added in 130 ml sulfuric acid, and then the solution was reacted for 12 hours at 180° C. After finishing the reaction, the solution was added slowly into ice water and cooled at room temperature and titrated at pH 5~6 by adding sodium carbonate. And then the particles produced by the reaction were filtered and dried under reduced pressure by using methanol.

The particles were purified using normal chromatography and obtained the particles of the compound b-1. (8.3 g, 35.6%)
R_{f} = 0.25 (Silicagel, isobutanol/n-propanol/ethyl acetate/water 2:4:1:3 v/v/v/v)
LC/MS, calculated value of C₂₁H₁₆N₂O₅ 376.37, measured value of 375.05

### Synthesis of Compound B-2

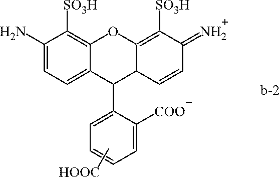

The compound b-1 (500 mg, 1.33 mmol, 1 eq) was added into 8 ml of 30% Fuming sulfuric acid and then the solution was stirred in 12 hours at 0° C. The reaction particles were captured by cold solution of 100 ml 1,4-dioxane and diethylether(1:2) and filtered by diatomite.

Then the particles were dissolved in methanol and titrated at pH 10 by triethylamine. Then the particles produced by the reaction were filtered and dried under reduced pressure and the particles were purified using reverse chromatography and obtained the particles of the compound b-2. (170 mg, 24%)
R_{f} = 0.11 (Silicagel, isobutanol/n-propanol/ethyl acetate/water 2:4:1:3 v/v/v/v)
LC/MS, calculated value of C₂₁H₁₆N₂O₁₁S₂ 536.48, measured value of 534.26

### Synthesis of Compound B-3

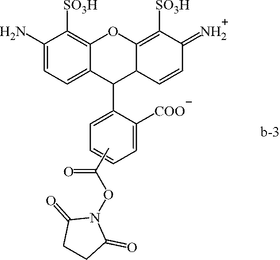

The compound b-2 (50 mg, 0.0935 mmol, 1 eq), N,N,N′,N′-Tetramethyl—O—(N-succinimidyl)uronium tetrafluoroborate (TSTU, 30.5 mg, 0.1589 mmol, 1.7 eq, TCI) and triethylamine (TEA, 120 µl, 4.675 mmol, 5 eq, TCI) were dissolved into 10 ml dimethylformamide and reacted for 1 hour at room temperature. Then the reacted particles were captured by ethylacetate and dried under reduced pressure. The particles of b-3 were obtained (59 mg, 100%).
R_{f} = 0.45 (Silicagel, isobutanol/n-propanol/ethyl acetate/water 2:4:1:3 v/v/v/v)
LC/MS, calculated value of C₂₅H₁₉N₃O₁₃S₂ 633.56, measured value of 631.02

### Synthesis of Compound B-4

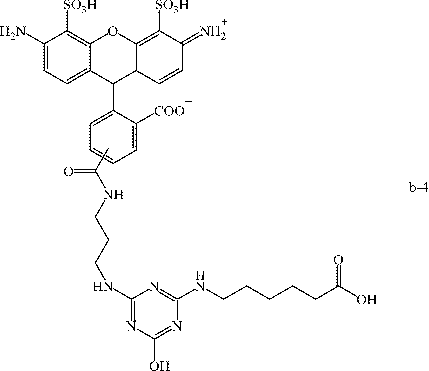

The compound b-3 (59 mg, 0.0935 mmol, 1 eq) and the compound a-3 (83.6 mg, 0.280 mmol, 3 eq) were added into 10 ml of distilled water and N,N-Diisopropylethylamine (DIPEA, 100 ul, 4.675 mmol, 5 eq) were slowly were added, The reaction was carried out for 12 hours at room temperature, and the reaction solution were lyophilized. The obtained solid particles were fully dissolved in distilled water. The pure compound of b-4 was obtained through RP-C18 reverse chromatography with acetonitrile solution (22.7 mg, 27.2%).
R_{f} = 0.44 (Silicagel, isobutanol/n-propanol/ethyl acetate/water 2:4:1:3 v/v/v/v)
LC/MS, calculated value of C₃₃H₃₆N₈O₁₃S₂ 816.81, measured value of 815.26

### Synthesis of Compound 1

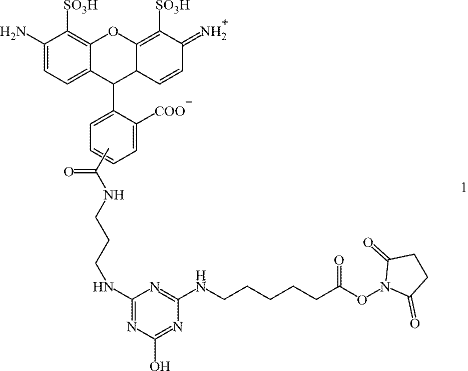

The compound b-4 (50 mg, 0.094 mmol, 1 eq), N,N,N′,N′-Tetramethyl—O—(N-succinimidyl)uronium tetrafluoroborate (TSTU, 30.5 mg, 0.159 mmol, 1.7 eq, TCI) and triethylamine (TEA, 120 µl, 0.468 mmol, 5 eq, TCI) were dissolved into 10 ml dimethylformamide and reacted for 1 hour at room temperature. Then the reacted particles of compound 1 were captured by ethylacetate and dried under reduced pressure. The pure compound of compound 1 was obtained through RP-C18 reverse chromatography with acetonitrile solution (21 mg, 35.6%).
Rf = 0.53 (Silicagel, isobutanol/n-propanol/ethyl acetate/water 2:4:1:3 v/v/v/v)
LC/MS, calculated value of C₂₅H₁₉N₃O₁₃S₂ 633.56, measured value of 631.02

### Example 3: Synthesis of Compound 2 With Exemplary Embodiments of the Present Invention

### Synthesis of Compound C-1

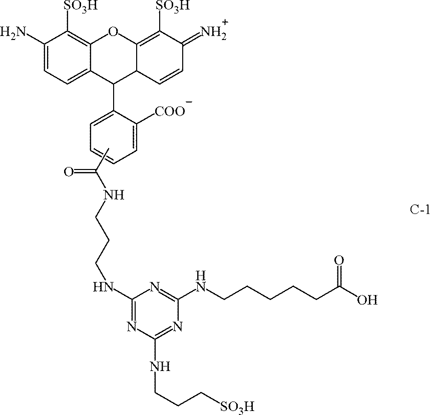

The compound b-3 (200 mg, 0.238 mmol, 1 eq) and the compound a-4 (174 mg, 0.415 mmol, 1.8 eq) were added into 20 ml of DMF, and N,N-Diisopropylethylamine (DIPEA, 0.4 ml, 1.19 mmol, 5 eq) was slowly added. The reaction was carried out for 12 hours at room temperature, and the reaction solution were lyophilized. The obtained solid particles were fully dissolved in distilled water. The pure compound of c-1 was obtained through RP-C18 reverse chromatography with acetonitrile solution (130 mg, 58.3%).
Rf = 0.44 (Silicagel, isobutanol/n-propanol/ethyl acetate/water 2:4:1:3 v/v/v/v)
LC/MS, calculated value of C₃₆H₄₃N₉O₁₅S₃ 937.97, measured value of 935.18

### Synthesis of Compound 2

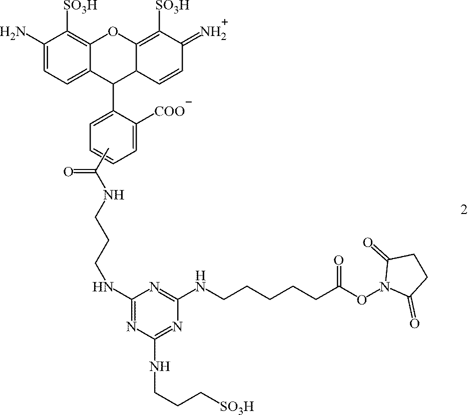

The compound c-1 (130 mg, 0.139 mmol, 1 eq), N,N,N′,N′-Tetramethyl—O—(N-succinimidyl)uronium tetrafluoroborate (TSTU, 54.5 mg, 0.181 mmol, 1.3 eq, TCI) and triethylamine (TEA, 0.096 ml, 0.696 mmol, 5 eq, TCI) were dissolved into 10 ml dimethylformamide and reacted for 1 hour at room temperature. Then the reacted particles of compound 1 were captured by ethylacetate and dried under reduced pressure. The pure compound of compound 2 was obtained (13.7 mg, 14.2%).
Rf = 0.52 (Silicagel, isobutanol/n-propanol/ethyl acetate/water 2:4:1:3 v/v/v/v)
LC/MS, calculated value of C₄₀H₄₆N₁₀O₁₇S₃ 1035.04, measured value of 1034.48

### Example 4: Synthesis of Compound 3 With Exemplary Embodiments of the Present Invention

### Synthesis of Compound D-1

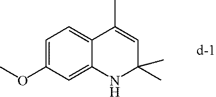

m-Anisidine (24.6 g, 0.2 mol, 1 eq, Sigma-Aldrich) and p-toluenesulfonic acid 2.6 g were added into 200 ml cyclohexane and dispersed.

The reaction solution was heated until 80~90° C. and then 42 ml aceton was trickled into the solution during 8~10 hours and the solution were stirred for a day and night at constant temperature.

After the reaction, the solution was cooled until 70° C. and then soda ash solution was added into the solution and stirred in 2 hours at room temperature. The soda ash solution was prepared from 0.6 g soda ash and 20 ml distilled water.

The reaction solution was extracted by distilled water and the organic layer was collected and dried under reduced pressure.

The reaction solution was washed through MC silicagel chromatogrphy and the compound d-1 was obtained (19.2 g, 43 %).

Rf = 0.65 (Silicagel, MC)

### Synthesis of Compound D-2

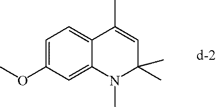

The compound d-1 (78.3 mmol, 1 eq) was added into iodomethane (166 g, 1.174 mol, 15 eq) and the reaction was proceeded under heated reflux condition in 12 hours. After the reaction, the solution was dried under reduced pressure and the iodomethane was removed.

The reaction solution was washed through MC silicagel chromatogrphy and the compound d-2 was obtained (14.1 g, 83 %).

Rf = 0.8 (Silicagel, MC)

### Synthesis of Compound D-3

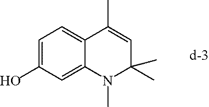

The compound d-2 (14.11 g, 64.9 mmol) was added into a mixture solution of 50 ml acetic acid and 50 ml bromic acid and the reaction was proceeded under heated reflux condition. The reaction solution was accoled at room temperature and 300 ml MC and 200 ml distilled water was added into the solution, The solution was stirred in 30 minutes. And then the solution was titrated through 30% caustic soda.

The reaction solution was extracted and the organic layer was collected and dried under reduced pressure. Finally the compound d-3 ws obtained (11.4 g, 86 %).

Rf = 0.2 (Silicagel, MC)

### Synthesis of Compound D-4

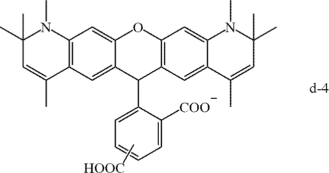

The compound d-3 (11.4 g, 56.1 mmol, 2 eq), trimellitic anhydride (5.38 g, 28 mmol, 1 eq) and 1 g of p-toluenesulfonic acid were added into 60 ml propionic acid and the reaction was proceeded under heated reflux condition in 12 hours.

After the reaction, the solution was dried under reduced pressure and the solvent was removed. The reaction solution was washed through MC silicagel chromatogrphy and the compound d-4 was obtained (6.4 g, 41 %).
Rf= 0.2 (RP-C18, acetonitrile/water 1:1 v/v)
LC/MS, calculated value of C₃₅H₃₅N₂O₅ 563.66, measured value of 567.0

### Synthesis of Compound D-5

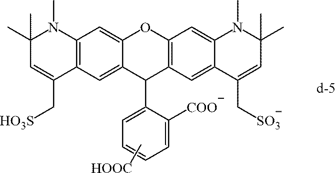

The compound d-4 (3 g, 5.32 mmol) was added into 40 ml strong sulfuric acid. After the solution was cooled at 0° C., the reaction was carried out for 2 hours. Then the reaction was more carried out for 12 hours at room temperature.

After the reaction, 60 ml dioxane was added and later the reaction particles were produced by adding 2 L diethylether.

The solution was filtered by Büchner funnel filled with diatomite and the filtered particles were dispersed in water and titrated by adding sodium bicarbonate. The reaction solution was filtered by Büchner funnel and the filtrate was dried under reduced pressure. The filtrate was purified by sillicagel chromatography and the compound d-5 was obtained by reduced pressure drying (610 mg, 16 %).
R_{f}= 0.6 (RP-C18, acetonitrile/water 1:5 v/v)
LC/MS, calculated value of C₃₅H₃₃N₂O₁₁S₂ 721.77, measured value of 720.6

### Synthesis of Compound D-6

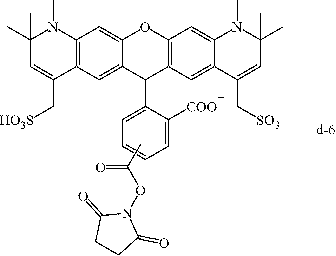

The compound d-5 (250 mg, 0.346 mmol, 2 eq) was added into a mixture of DMF (12 ml) and DW (5 ml) and fully dissolved.

After the solution was cooled at 0° C., O—(N-Succinimidyl)-N,N,N′,N′-tetramethyluronium tetrafluoroborate (TSTU, 312 mg, 1.038 mmol, 3 eq, Sigma-Aldrich) was added and the solution was stirred in 2 hours. After the reaction was finished, the reaction particles were lyophilized and purified by reverse chromatography using acetonitrile and water and obtained (40 mg, 14 %).
R_{f}= 0.4 (RP-C18, acetonitrile/water 1:3 v/v)
LC/MS, calculated value of C₃₉H₃₈N₃O₁₃S₂ 820.86, measured value of 817.9

### Synthesis of Compound D-7

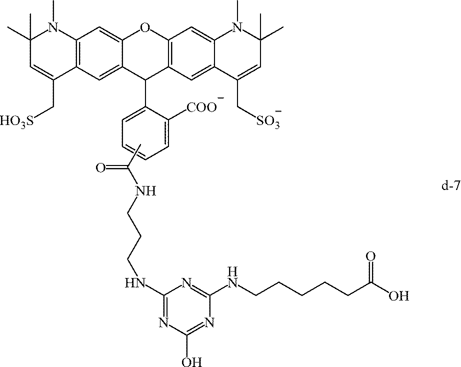

The compound d-6 (400 mg, 0.488 mmol, 1 eq), and the compound a-3 (592 mg, 1.952 mmol, 4 eq) were added into 60 ml of water and N,N-Diisopropylethylamine (DIPEA, 850 ul, 4.88 mmol, 10 eq) were slowly were added. The reaction was carried out for 12 hours at room temperature and the reaction solution were lyophilized. The obtained solid particles were fullydissolved in distilled water. The pure compound of d-7 was obtained through RP-C18 reverse chromatography with acetonitrile solution (374 mg, 76.6%).
Rf = 0.4 (Silicagel, isobutanol/n-propanol/ethyl acetate/water 2:4:1:3 v/v/v/v)
LC/MS, calculated value of C₄₇H₅₄N₈O₁₃S₂ 1003.1, measured value of 1002.0

### Synthesis of Compound 3

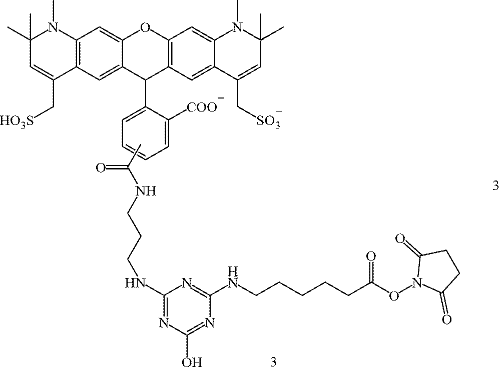

The compound d-7 (110 mg, 0.11 mmol, 1 eq), N,N,N′,N′-Tetramethyl—O—(N-succinimidyl)uronium tetrafluoroborate (TSTU, 40 mg, 0.13 mmol, 1.2 eq, TCI) and triethylamine (TEA, 55 µl, 0.40 mmol, 3 eq, TCI) were slowly mixed and reacted for 1 hour at room temperature.

After the reaction solution were lyophilized, the solid particles of compound 3 were purified through RP-C18 reverse chromatography with acetonitrile solution (374 mg, 76.6%) and obtained (65 mg, 53.7 %).
Rf = 0.45 (Silicagel, isobutanol/n-propanol/ethyl acetate/water 2:4:1:3 v/v/v/v)
LC/MS, calculated value of C₅₁H₅₇N₉O₁₅S₂ 1100.2, measured value of 1099.1

### Example 4: Synthesis of Compound 4 With Exemplary Embodiments of the Present Invention

### Synthesis of Compound E-1

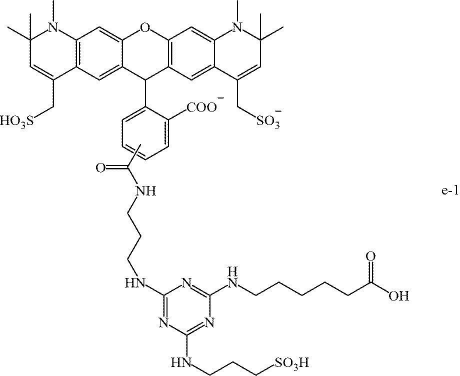

The compound d-6 (50 mg, 0.061 mmol, 1 eq), and the compound a-4 (38 mg, 0.092 mmol, 1.5 eq) were added into 10 ml DMF and N,N-Diisopropylethylamine (DIPEA, 0.11 ml, 0.61 mmol, 10 eq) were slowly added. The reaction was carried out for 12 hours at room temperature and the reaction solution were lyophilized. Then the reacted particles were captured by ethylacetate and dried under reduced pressure. The pure compound of e-1 was obtained through RP-C18 reverse chromatography with acetonitrile solution (56 mg, 81.7 %).
Rf = 0.35 (Silicagel, isobutanol/n-propanol/ethyl acetate/water 2:4:1:3 v/v/v/v)
LC/MS, calculated value of C₅₀H₆₁N₉O₁₅S₃ 1124.3, measured value of 1123.1

### Synthesis of Compound 4

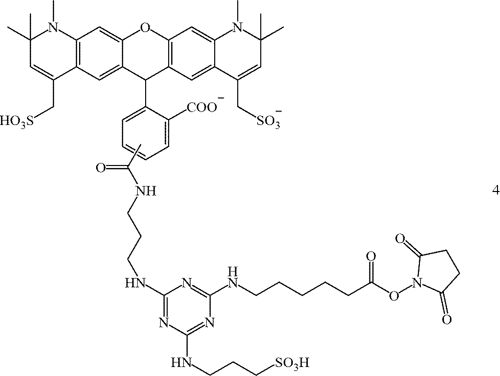

The compound e-1 (50 mg, 44.47 umol, 1 eq), N,N,N′,N′-Tetramethyl—O—(N-succinimidyl)uronium tetrafluoroborate (TSTU, 16 mg, 53.37 umol, 1.2 eq, TCI) and triethylamine (TEA, 9 mg, 88.94 mmol, 2 eq, TCI) were dissolved into 10 ml dimethylformamide and reacted for 1 hour at room temperature. Then the reacted particles were captured by ethylacetate and dried under reduced pressure. The pure compound of compound 4 was obtained (28 mg, 51.6 %).
Rf = 0.40 (Silicagel, isobutanol/n-propanol/ethyl acetate/water 2:4:1:3 v/v/v/v)
LC/MS, calculated value of C₅₄H₆₄N₁₀O₁₇S₃ 1221.3, measured value of 1220.2

Fluorescent values at the same absorption wavelength of the compounds of exemplary embodiments of the present invention and the conventional compound not included in exemplary embodiments of the present invention were compared through Comparative Examples. Thereafter, the usability of the present invention in detection biomaterials was validated.

### Example 5. Estimation of Optical Characteristics of Compound 2

### Investigation of the Characteristics of Absorption and Fluorescence

The absorption and fluorescence characteristics of the compound 2 in accordance with embodiments of the present invention and a conventional dye(Alexa Flour™ 488 NHS ester)were analyzed and verified by the analysis

Firstly, the said two fluorescent dyes were dissolved in DMF and the stock solution were prepared (10 mg/ml) and the pH of the stock was maintained 7.4 and diluted by 10 mM phosphate buffered saline(1X PBS). Then the analysis were proceeded. The absorption were measured by Cary 3500 UV-Vis photometer and the fluorescence were measured by LS 55 fluorescence spectrometer(PerkinElmer).

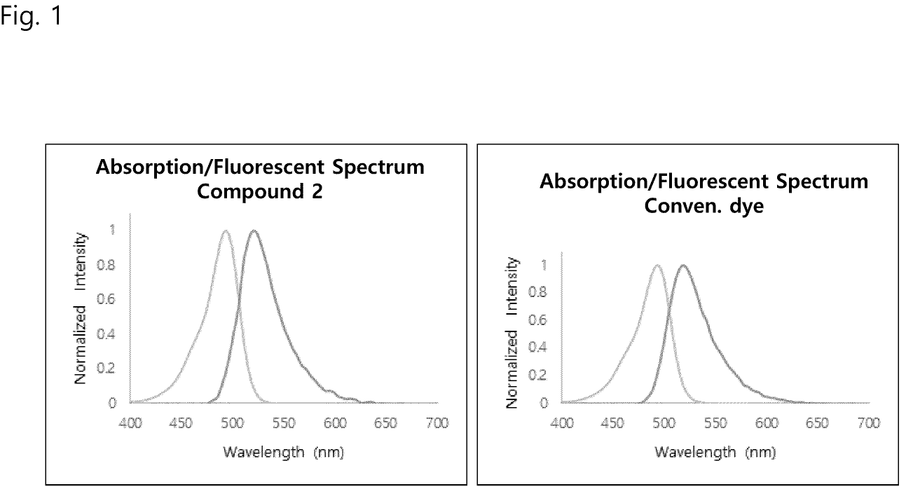

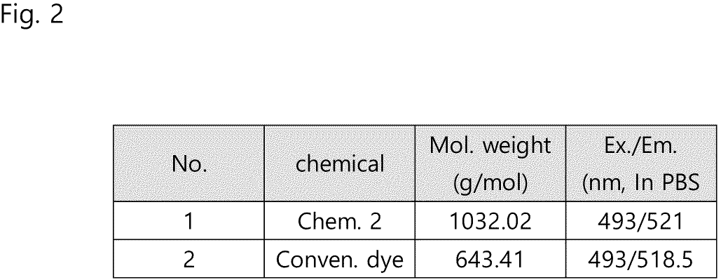

FIG. 1 shows the absorption and fluorescence spectrum of the two dyes, and FIG. 2 represents the optical characteristics of the dyes. From the results, it is verified that the compound 2 in accordance with embodiments of the present invention has similar optical characteristics as those of the conventional dye.

### Comparison of Performance After Protein Labeling

After antibody-labeling (Invitrogen, Goat anti Rabbit IgG H+L Secondary Ab, 150 kDa) of the compound 2 in accordance with embodiments of the present invention and a conventional dye(Alexa Fluor™ 488 NHS Ester), the labeling ratio(F/P molar ratio) were measured and compared.

Before the labeling, the compound 2 and the conventional dye were dissolved in DMF and the stock solution of 10 mg/ml was prepared. Thereafter the dyes were reacted with 0.1 mg of the antibody by each reaction ratio(2, 5, 15, 25 Fold). The reaction buffer was prepared with the final pH of 8.5, and the final concentration of the antibody was 2 mg/ml. The reaction was carried out for 1 hour in dark environment at room temperature through stirring. And the reaction particles were separated and obtained through the column filled with Sephadex G-25 resin(Cytiva). The resin was used in a buffer equilibrium with 1X PBS.

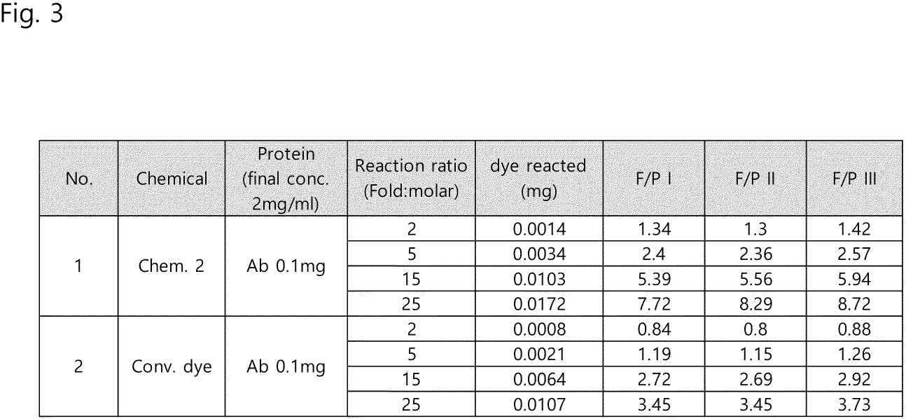

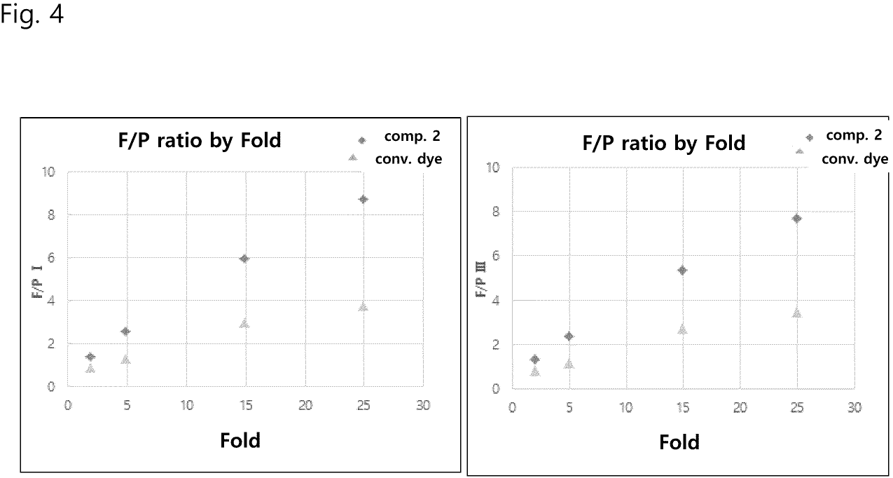

The absorption intensity of each reaction particle was measured at the wavelength of 280, 493 nm respectively(Agilent, Cary 3500 UV-Vis spectrophotometer), and the labeling ratio was calculated through a universal formula. FIG. 3 shows the result of labeling ratio by the amount of reacted-dye and FIG. 4 shows the graph of the results simultaneously.

In FIG. 3, F/P I was calculated by the ratio of the conventional dye described in the website (Extinction coefficient : 71,000, Correction factor : 0.11), and F/P II was calculated by the actual analyzed value (Extinction coefficient : 75,000, Correction factor : 0.154) and F/P III is the value of the compound 2 calculated by the actual analyzed value (Extinction coefficient : 68,000, Correction factor : 0.138).

Because the F/P I and F/P II are similar to each other, it was compared F/P I and F/P II below.

Comparing the results, the compound 2 shows higher labeling ratio than the conventional dye at the same molar condition.

In addition, it is verified that the F/P ratio of compound 2 at 15 Fold (0.0103 mg of the compound 2 dissolved) is higher than that of the conventional dye at 25 Fold (0.0107 mg of the conventional dye dissolved).

FIG. 4, which shows the labeling ratios of the dyes by the amount of reacted-dye verifies that the compound 2 is more specific than the conventional dye.

### Comparison of the Fluorescent Intensities Between Chemical and Antibody Conjugates

By using the conjugates in the comparison experiment 5-(2), the fluorescent intensities of the two dyes after protein-labeling were compared.

The conjugates were used without dilution and the intensity was measured at Excitation 493 nm by LS 55 Fluorescence spectrometer of PerkinElmer.

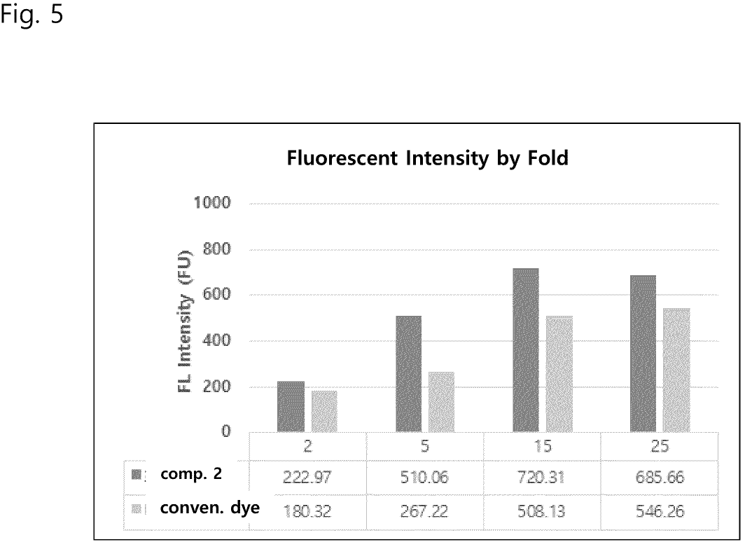

The results were shown in FIG. 5.

FIG. 5 shows that the fluorescent intensity of the compound 2-antibody conjugate is higher than that of the conventional dye-antibody conjugate in all of the labeling ratio(reaction ratio).

It is likely that the compound 2 shows higher fluorescent intensity compared to the conventional dye even if it is a less reaction ratio.

### Comparison of Fluorescent Intensity by Labeling Ratio

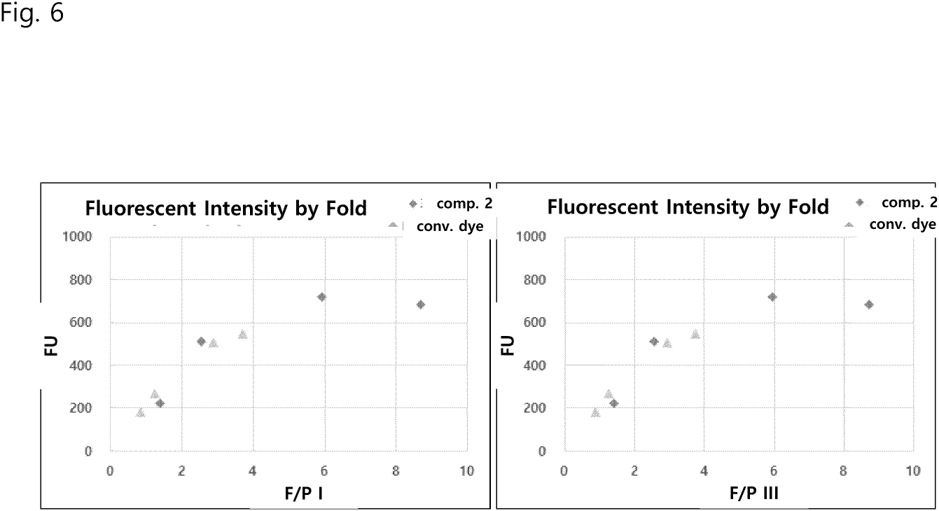

In FIG. 6, the fluorescent intensities of the two dyes by the labeling ratio were shown by measuring the fluorescent intensity by the reaction ratio in the comparison experiment 6-(2) and 6-(3).

Form the result, when the compound 2 and the conventional dye show similar labeling ratio, the fluorescent intensity of each dye looks similar. In addition, it is inferred that even the less amount of the compound 2 is used, the labeling ratio and the fluorescent intensity of compound 2 are similar level of the conventional dye or reach the level faster than the conventional dye in more amount.

### Example 6. Estimation of Optical Characteristics of Compound 4

### Investigation of the Characteristics of Absorption and Fluorescence

The absorption and fluorescence characteristics of the compound 4 in accordance with embodiments of the present invention and a conventional dye(Alexa Flour™ 594 NHS ester)were analyzed and verified by the analysis

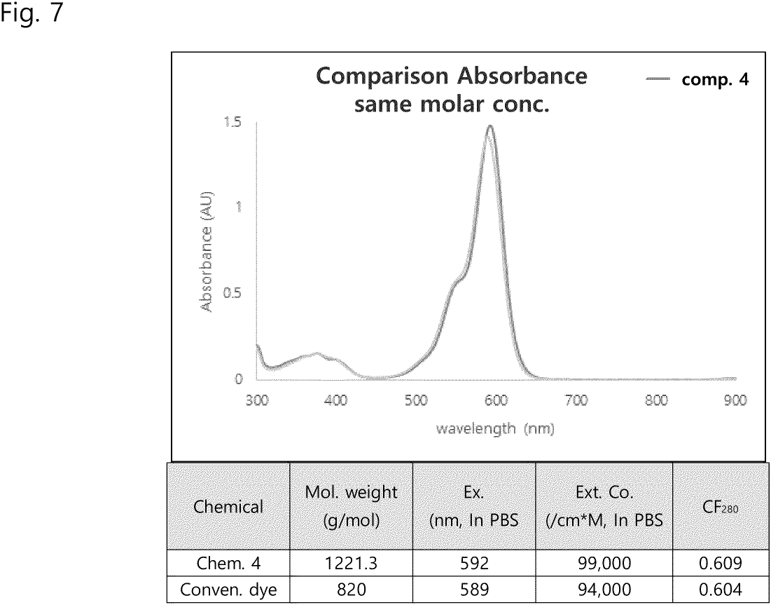

First, the said two fluorescent dyes were dissolved in DMF, and the stock solution was prepared (10 mg/ml). The pH of the stock solution was maintained 7.4 and diluted by 10 mM phosphate buffered saline(1X PBS), and the concentration of each dye solution was 15 uM. Then the absorption intensity was measured. The measurement was done with Cary 3500 UV-Vis photometer, and the results were shown in FIG. 7.

FIG. 7 shows that the two dyes show similar absorption spectrum. In absorption intensity and molar absorption coefficient, the compound 4 is higher than the conventional dye.

### The Comparison of Fluorescent Characteristics and Intensity

The fluorescent characteristics and intensity of the compound 4 and the conventional dye were compared.

Each stock solution was prepared by adding the two dyes in DMF. The concentrations of the stock solutions were the same as 10 mg/ml.

First, to compare the fluorescent intensity at the same molar concentration, the stock solutions were diluted by 1X PBS until the concentrations reach 0.005 uM and then the fluorescence were measured at the excitation wavelength (nm) of the dyes.

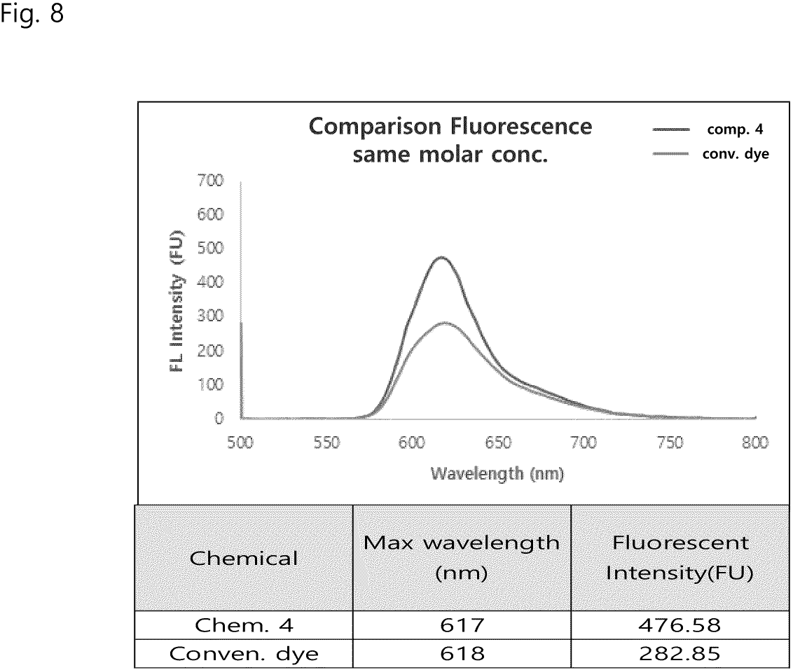

The fluorescence was measured by LS 55 Fluorescence spectrometer (PerkinElmer), and the results were shown in FIG. 8.

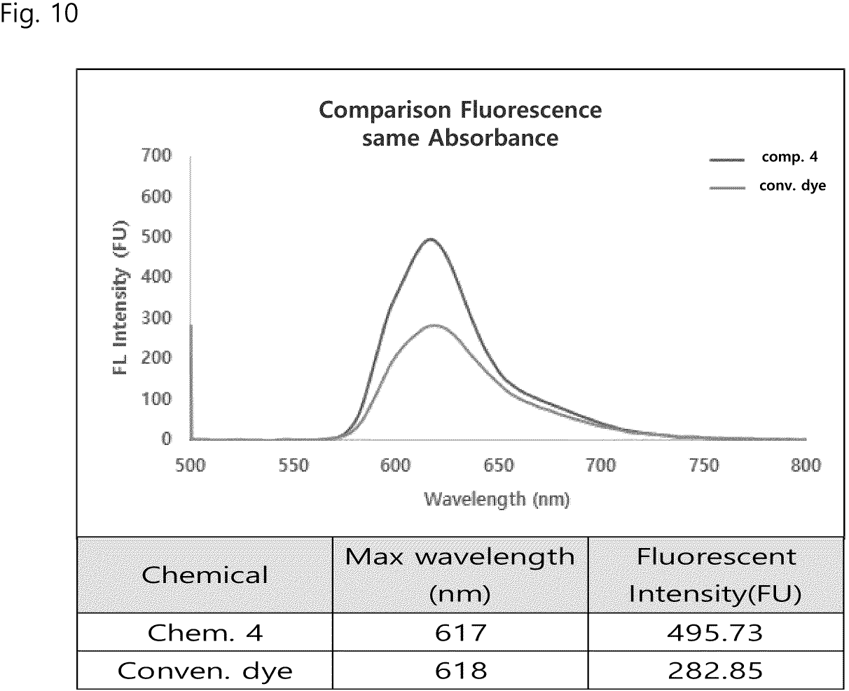

Thereafter, to compare the fluorescent intensity of each dye in the same weight concentration, the stock solutions of the two dyes were diluted based on the equivalent amount from the absorption result, and the fluorescence were measured. The results were shown in FIG. 10.

From the results, it seems likely that the fluorescent intensity of the compound 4 is higher than the conventional dye both in the same molar concentration and in the same weight concentration. The maximum fluorescent wavelengths of the compound 4 and the conventional dye in the solvent 1X PBS are 617 nm and 618 nm respectively. The maximum wavelengths are similar.

### Comparison of Performance After Protein Labeling

After antibody-labeling (Invitrogen, Goat anti Rabbit IgG H+L Secondary Ab, 150 kDa) of the compound 4 in accordance with embodiments of the present invention and a conventional dye(Alexa Fluor™ 594 NHS Ester), the labeling ratios(F/P molar ratio) were measured and compared.

Before the labeling, the compound 4 and the conventional dye were dissolved in DMF and the stock solution of 10 mg/ml was prepared. Thereafter the dyes were reacted with 0.1 mg of the antibody by each reaction ratio(2, 5, 15, 25 Fold). The reaction buffer was prepared with the final pH of 8.3~8.5, and the final concentration of the antibody was 2 mg/ml. The reaction was carried out for 1 hour in dark environment at room temperature through stirring. And the reaction particles were separated and obtained through the column filled with Sephadex G-25 resin(Cytiva). The resin was used in a buffer equilibrium with 1X PBS.

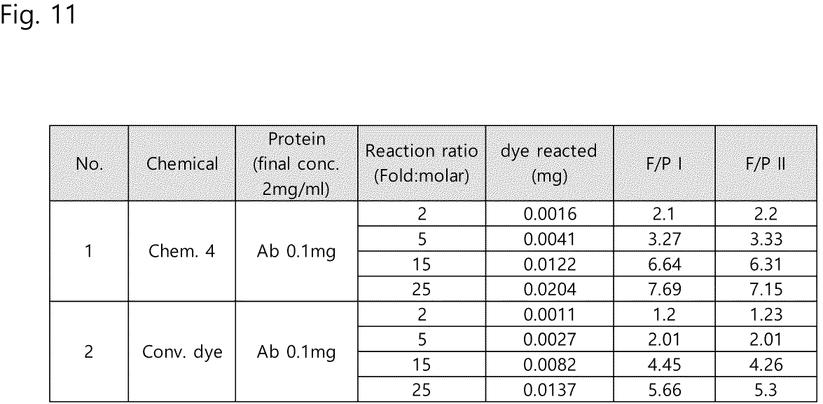

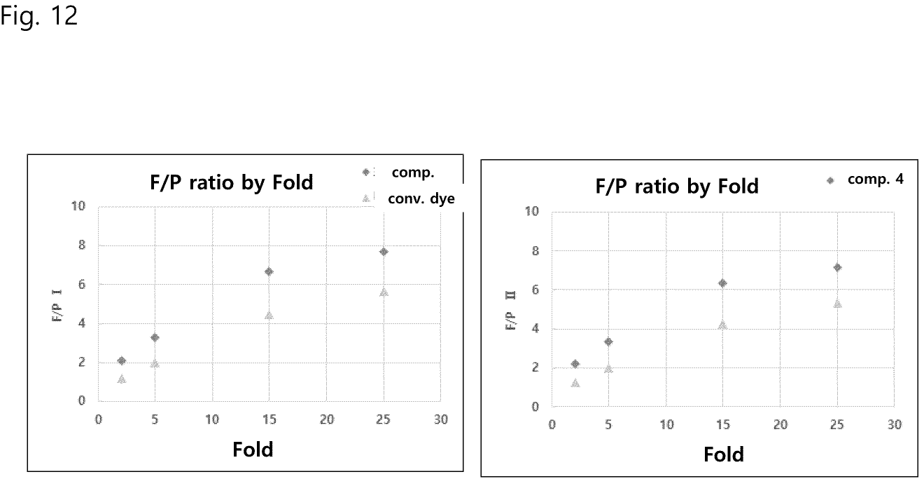

The absorption intensity of each reaction particle was measured at the wavelength of 280, 590 nm respectively(Agilent, Cary 3500 UV-Vis spectrophotometer) and the labeling ratio was calculated through a universal formula. FIG. 11 shows the result of labeling ratio by the amount of reacted-dye, and FIG. 12 shows the graph of the results simultaneously.

In FIG. 11, F/P I was calculated by the analyzed ratios of the dyes (compound 4: Extinction coefficient : 99,000, Correction factor : 0.609 / conventional dye : Extinction coefficient : 94,000, Correction factor : 0.604), F/P II was calculated by the ratio of the conventional dye described in the website (Extinction coefficient : 90,000, Correction factor : 0.56).

Comparing the results, the compound 4 shows a higher labeling ratio than the conventional dye at the same molar condition.

In addition, comparing the point at which similar amounts of dyes are used (compound 4 0.0016 mg/conventional dye 0.0011 mg, compound 4 0.0122 mg/conventional dye 0.0137 mg), the compound 4 shows higher labeling ratio than the conventional dye. These results suggest that the compound 4 is more specific than the conventional dye.

### Comparison of the Fluorescent Intensities Between Chemical and Antibody Conjugates

By using the conjugates in the comparison experiment 6-(3), the fluorescent intensities of the two dyes after protein-labeling was compared.

The intensity was measured at Excitation 590 nm by LS 55 Fluorescence spectrometer of PerkinElmer.

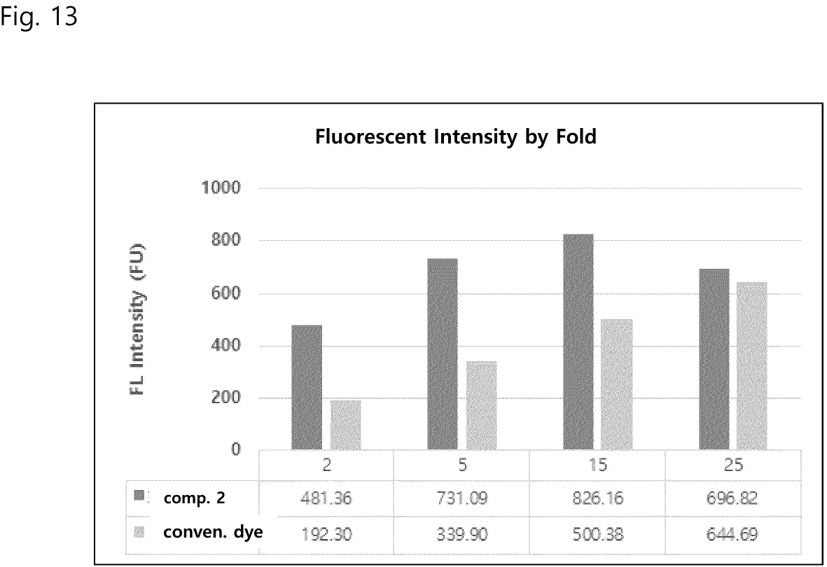

The results were shown in FIG. 13.

FIG. 13 shows that the fluorescent intensity of the compound 4-antibody conjugate is higher than that of the conventional dye-antibody conjugate in all of the labeling ratio(reaction ratio).

It is likely that the compound 4 shows higher fluorescent intensity compared to the conventional dye even if it is relatively less reaction ratio.

### Comparison of Fluorescent Intensity by Labeling Ratio

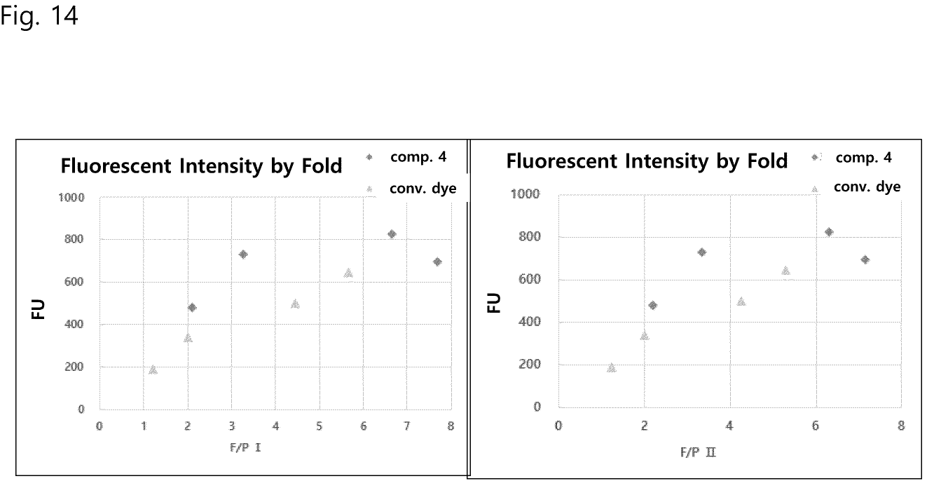

In FIG. 14, the fluorescent intensities of the two dyes by the labeling ratio were shown by measuring the fluorescent intensity by the reaction ratio in the said comparison experiment 6-(3) and 6-(4).

Form the result, when the compound 4 and the conventional dye show similar labeling ratio (for example, the ratio is 2), the fluorescent intensity of the compound 4 shows higher than the conventional dye. It is likely that even the less amount of the compound 4 is used, the labeling ratio and the fluorescent intensity of compound 4 are similar level of the conventional dye or reach the level faster than the conventional dye in more amount.

### Testing Example 1 : Comparing Performance of Novel Fluorescent-Protein Conjugates

### Preparing Novel Fluorescent-Protein Conjugates

For testing the performance of novel fluorescent, novel fluorescents of FSD 488(sulfone) and FSD 594(sulfone) in accordance with embodiments of the present invention and conventional dyes(FSD 488(Cl). FSD 594(Cl), Alexa 488, Alexa 594) are reacted with antibody (Thermo, Goat anti-Mouse IgG H+L Secondary Ab, 140 kDa).

The six dyes were labelled at the labeling ratio of 5-6(Degree of Labeling, DOL) and reaction buffer were prepared at the pH 8.5 by using 10 mM pH 7.4 Phosphate buffered saline (1X PBS) and 1 M pH 9.4 Sodium carbonate-Bicarbonate buffer (1 M CBC).

The labeling ratio was calculated through the universal formula in absorption intensity.

### Testing Performance Through Immunofluorescence After Labeling

For testing the performance of the conjugates in testing 1-(1), the immunofluorescence staining was carried out.

For the analysis, HeLa cells and alpha tubulin monoclonal antibody (Thermo) as the antibody were used.

The first antibody was treated with the concentration of 1:500, and the second antibody was treated with 1:2000. And, the reaction was carried out for 1 hour at room temperature.

For the fluorescent analysis, Nikon ECLIPSE Ti-U was used.

FSD 488(Sulfone, Cl) and Alexa 488 were analyzed by FITC filter, and FSD 594(Sufone, Cl) and Alexa 594 were analyzed by TRITC filter.

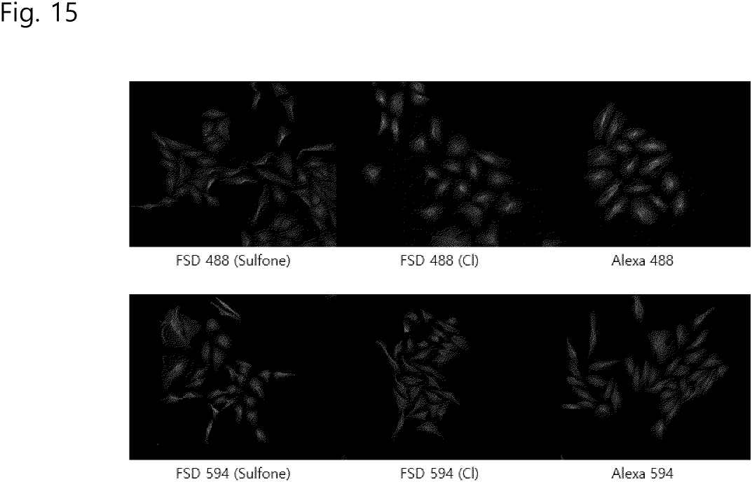

From the results shown in FIG. 15, the novel fluorescents and the conventional dyes reacted specifically without non-specific reaction and shown similar fluorescent intensity each other.

As described above, the fluorescent compound introducing a triazine-substituted benzene linker to a rhodamine-based fluorescent compounds provided in exemplary embodiments of the present invention has high fluorescence efficiency such as fluorescent intensity and the like as compared with the conventional fluorescent compound at the same concentration to accurately detect a target material even in a small amount of biomaterial.

The present invention is not limited by the above-described embodiments, and various modifications and changes can be made by those skilled in the art and may be used in various biological and chemical fields, and are included in the spirit and scope of the present invention as defined in the appended claims.

1. A fluorescent compound for detecting a biomaterial having the following Chemical Formula 1: 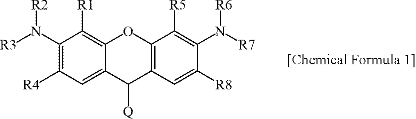
In Chemical Formula 1 above,
R₁ and R₅ are the same as or different from each other, and each independently selected from H, —SO₃⁻ and SO₃H,
R₂ and R₆ are the same as each other or each independently selected from hydrogen or methyl
R₄ and Rs are the same as or different from each other, and each independently selected from H, —SO₃⁻ and (CH₂)SO₃H,
R₃ and R₄ can form alkylene chain with arbituary-substituted 3 carbons combined with the nitrogen atom attached with R₃,
R₇ and R₈ can form alkylene chain with arbituary-substituted 3 carbons combined with the nitrogen atom attached with R₇,
when R₈ and R₈ do not form alkylene chain, R₆, R₇ and R₈ are hydrogen at the same time and the nitrogen atom attached with R₆ and R₇ at the same time forms double bond with the carbon of mother molecule and has positive charge,
the said Q is a substituent having a chemical structure below 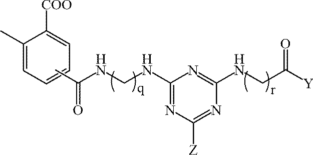
Z is OH or NH(CH₂)ₛSO₃H,
q is an integer of 0 to 10,
r is an integer of 1 to 10, and
s is an integer of 1 to 7,
Y is selected from H, an N-succinimidol group, a hydrazinyl group, an N-hydroxysuccinimidyl group, an N-hydroxysuccinimidyl oxy group, a sulfosuccinimidyl oxy, a 4-sulfo-2,3,4,5-tetrafluoro phenyl group, a maleicimide C₀₋₁₀ alkylamyl group, a vinylsulfonyl group, a vinylsulfonyl C₀₋₆ alkylaminyl group and an amino C₀₋₆ alkyl.

2. The fluorescent compound of claim 1, wherein the compound of Chemical Formula 1 above is any one selected from compounds represented by the following Chemical Formulas.

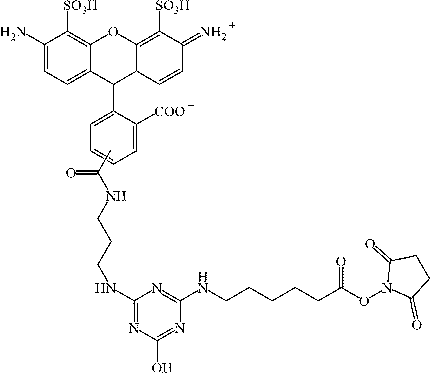

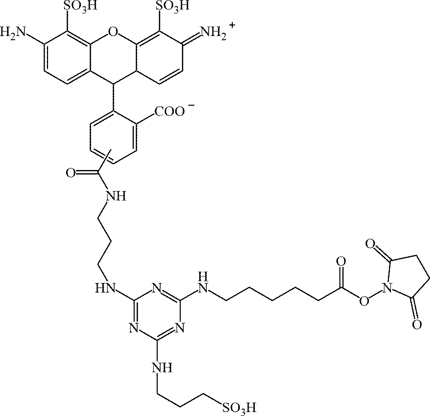

3. The fluorescent compound of claim 1, wherein the compound of Chemical Formula 1 above is any one selected from compounds represented by the following Chemical Formulas.

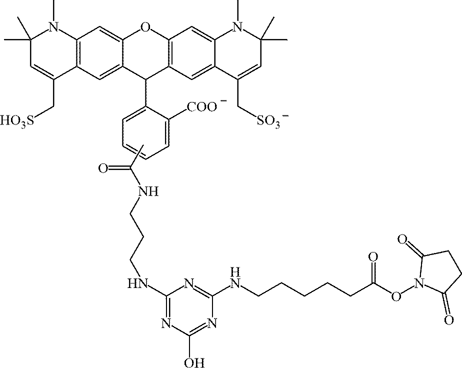

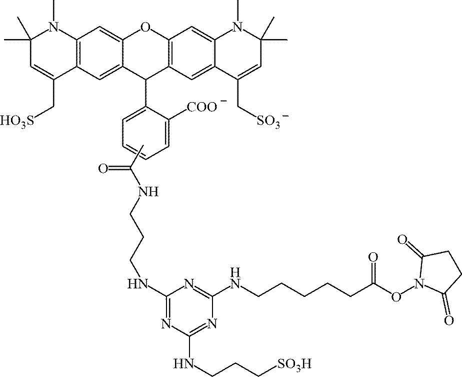

4. The fluorescent compound of claim 1, wherein the biomaterial is any one selected from the group consisting of proteins, peptides, carbohydrates, sugars, fats, antibodies, proteoglycan, glycoprotein, and siRNA.

5. A fluorescent diagnostic composition for detecting a biomaterial comprising a fluorescent compound represented by the following Chemical Formula 1: 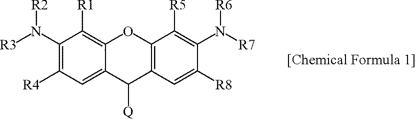
In Chemical Formula 1 above,
R₁ and R₅ are the same as or different from each other, and each independently selected from H, —SO₃⁻ and SO₃H,
R₂ and R₆ are the same as each other or each independently selected from hydrogen or methyl
R₄ and R₈ are the same as or different from each other, and each independently selected from H, —SO₃⁻ and (CH₂)SO₃H,
R₃ and R₄ can form alkylene chain with arbituary-substituted 3 carbons combined with the nitrogen atom attached with R₃,
R₇ and R₈ can form alkylene chain with arbituary-substituted 3 carbons combined with the nitrogen atom attached with R₇,
when R₈ and R₈ do not form alkylene chain, R₆, R₇ and R₈ are hydrogen at the same time and the nitrogen atom attached with R₆ and R₇ at the same time forms double bond with the carbon of mother molecule and has positive charge,
the said Q is a substituent having a chemical structure below 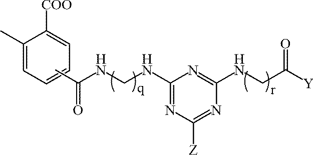
Z is OH or NH(CH₂)ₛSO₃H,
q is an integer of 0 to 10,
r is an integer of 1 to 10, and
s is an integer of 1 to 7,
Y is selected from H, an N-succinimidol group, a hydrazinyl group, an N-hydroxysuccinimidyl group, an N-hydroxysuccinimidyl oxy group, a sulfosuccinimidyl oxy, a 4-sulfo-2,3,4,5-tetrafluoro phenyl group, a maleicimide C₀₋₁₀ alkylamyl group, a vinylsulfonyl group, a vinylsulfonyl C₀₋₆ alkylaminyl group and an amino C₀₋₆ alkyl.

6. The fluorescent diagnostic composition of claim 5, wherein the compound of Chemical Formula 1 above is any one selected from compounds represented by the following Chemical Formulas.

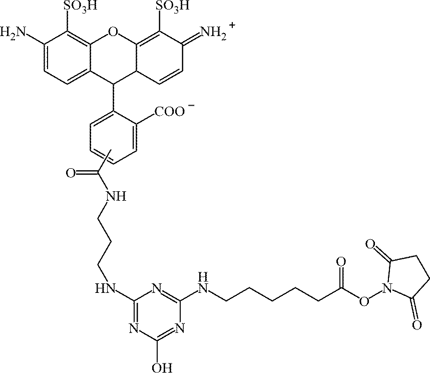

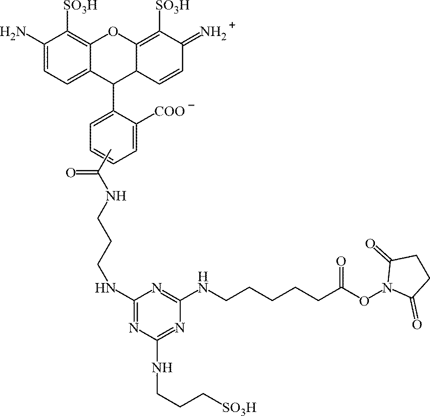

7. The fluorescent diagnostic composition of claim 5, wherein the compound of Chemical Formula 1 above is any one selected from compounds represented by the following Chemical Formulas.

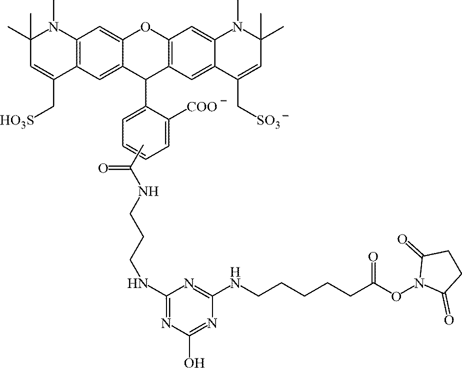

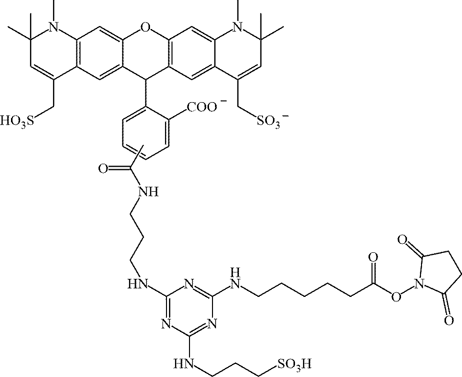

8. The fluorescent diagnostic composition of claim 5, wherein the biomaterial is any one selected from the group consisting of proteins, peptides, carbohydrates, sugars, fats, antibodies, proteoglycan, glycoprotein, and siRNA.
